(19) United States
(12) Patent Application Publication
(10) Pub. No.: US 2010/0261766 A1
(43) Pub. Date: Oct. 14, 2010

(54) Phenyl-Oxetanyl-Derivatives

(76) Inventors: Rainer Albert, Basel (CH); Nigel Graham Cooke, Basel (CH); Frederic Zecri, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/744,572

(22) PCT Filed: Dec. 1, 2008
(86) PCT No.: PCT/EP08/66517

§ 371 Date: May 25, 2010

(30) Foreign Application Priority Data
Nov. 30, 2007 (EP) 07122001.6

(51) Int. Cl. A61K 31/4245; C07D 413/10; C07D 305/10; C07D 305/08; A61P 29/00; A61P 37/06; A61P 31/00

(52) U.S. Cl. 514/364; 548/131; 549/511; 549/510

(57) ABSTRACT

Novel organic compounds are described and claimed which may in particular be useful in the treatment of diseases being triggered by the (human) autoimmune system.

The present invention relates to polycyclic compounds, processes for their production, their use as pharmaceuticals and to pharmaceutical compositions comprising them.

More particularly the present invention provides in a first aspect a compound of formula I and/or a pharmaceutical acceptable salt thereof.

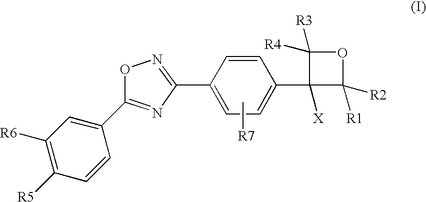

wherein
X is amino, alkylamino, hydroxyl, alkoxy or halo;
R1, R2, R3 and R4 are independently from each other H or lower alkyl;
R5 is alkoxy optionally substituted by halogen, C₃₋₆cycloalkyloxy optionally substituted by halogen;
R6 is cyano, acyl, alkyl optionally substituted by halogen, or alkyl substituted by alkoxy; and
R7 is H, lower alkyl optionally substituted by halogen, lower alkoxy optionally substituted by halogen, or halogen.

Halogen may be fluorine, chlorine or bromine, preferably fluorine or chlorine, more preferably fluorine.

Alkyl as a group or present in a group may be straight or branched and may contain up to 8 carbon atoms. Lower in the context with alkyl denote a group with up to 4 carbon atoms. Examples of alkyl are methyl, ethyl, propyl, i-propyl, butyl, sec-butyl, i-butyl, t-butyl, pentyl, hexyl, heptyl, octyl and the like.

Alkoxy as a group or present in a group may be straight or branched and may contain up to 8 carbon atoms. Lower in the context with alkoxy denote a group with up to 4 carbon atoms.

Examples of alkoxy are methoxy, ethoxy, propyloxy, i-propyloxy, butyloxy, sec-butyloxy, butyloxy, t-butyloxy, pentyloxy, hexyloxy, heptyloxy, octyloxy and the like.

Alkyl or alkoxy substituted by halogen may be C₁₋₈alkyl or C₁₋₈alkoxy substituted by 1 to 5 halogen, e.g. CF₃ or CF₃—CH₂—O—. C₁₋₈alkyl-haloC₁₋₈alkoxy may be haloC₁₋₈alkoxy further substituted by C₁₋₈alkyl, e.g. in position 1. The same may apply to the other groups.

Acyl as used herein is a radical R_{d}CO wherein R_{d} is H, C₁₋₈alkyl, C₃₋₆cycloalkyl, C₃₋₆cycloalkyloxy, C₁₋₆alkoxy, benzyl or benzyloxy.

Preferably acyl is C₁₋₆alkyl-CO, C₁₋₆alkoxy-CO, benzyloxy-CO or benzyl-CO, more preferably C₁₋₆alkyl-CO or C₁₋₄alkoxy-CO, particularly C₁₋₄alkyl-CO, C₁₋₄alkoxy-CO, t-butoxycarbonyl or acetyl.

Alkylamino, as used herein, represents an amine wherein one or two of the hydrogens of the amine are replaced by straight or branched alkyl having from 1 up to 8 carbon atoms inclusive. Preferred alkylamino is lower alkylamino. Examples of alkylamino include N-methylamino, N,N-dimethylamino, N-ethylamino, N,N-diethylamino, N-n-propylamino, N,N-di-n-propylamino, N-i-propylamino, N,N-di-i-propylamino, N-butylamino and the like.

The following significances are preferred independently, collectively or in any combination or sub-combination:
i) R1 is H or methyl;
ii) R2 is H or methyl;
iii) R3 is H or methyl;
iv) R4 is H or methyl;
v) R1 has the same meaning as R2;
yl) R1 has the same meaning as R3;
vii) R1, R2, R3 and R4 are all hydrogen;
viii) R5 is alkoxy optionally substituted by halogen, or C₃₋₆cycloalkyloxy optionally substituted by halogen, more preferably alkoxy or C₃₋₆cycloalkyloxy, even more preferably lower alkoxy;
ix) R6 is cyano or acyl, more preferably cyano;
x) R7 is hydrogen;
xi) X is amino;
xii) X is hydroxyl;
xiii) X is aminoalkyl;
xiv) X is fluoro;

The compounds of formula I may exist in free form or in salt form, e.g. pharmaceutically acceptable salts. It will be appreciated that the compounds described herein, in particular compounds of formula I may exist in the form of an isomer, e.g. an optical isomer. For example, R4 may comprise an asymmetric carbon atom e.g. when R4 is branched alkyl. Or in another example, in a compound of formula (I), the carbon atom to which the group X is attached, the said carbon atom may additionally contain 3 different (non-identical) ligands and hence may per se represent an asymmetric center.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the term “pharmaceutically acceptable salts” refers to salts that retain the biological effectiveness and properties of the compounds of this invention and, which are not biologically or otherwise undesirable. In many cases, the compounds of the present invention are capable of forming acid and/or base salts by virtue of the presence of amino and/or carboxyl groups or groups similar thereto. Pharmaceutically acceptable acid addition salts can be formed with inorganic acids and organic acids, e.g., acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

The present invention includes all pharmaceutically acceptable isotopically-labeled compounds of the invention, i.e. compounds of formula (I), wherein (1) one or more atoms are replaced by atoms having the same atomic number, but an atomic mass or mass number different from the atomic mass or mass number usually found in nature, and/or (2) the isotopic ratio of one or more atoms is different from the naturally occurring ratio.

Examples of isotopes suitable for inclusion in the compounds of the invention comprises isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²³I, nitrogen, such as ¹³N and ¹⁵N, oxygen, such as ¹⁶O, ¹⁷O and ¹⁸O, phosphorus, such as ³²P, and sulphur, such as ³⁵S.

Certain isotopically-labeled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula I that contain groups capable of acting as donors and/or acceptors for hydrogen bonds may be capable of forming co-crystals with suitable co-crystal formers. These co-crystals may be prepared from compounds of formula I by known co-crystal forming procedures. Such procedures include grinding, heating, co-subliming, co-melting, or contacting in solution compounds of formula I with the co-crystal former under crystallization conditions and isolating co-crystals thereby formed. Suitable co-crystal formers include those described in WO 2004/078163. Hence the invention further provides co-crystals comprising a compound of formula I.

### Synthesis

The present invention further pertains to a process for the manufacture a compound in accordance to general formula (I), wherein the variables are as defined above (see appended scheme).

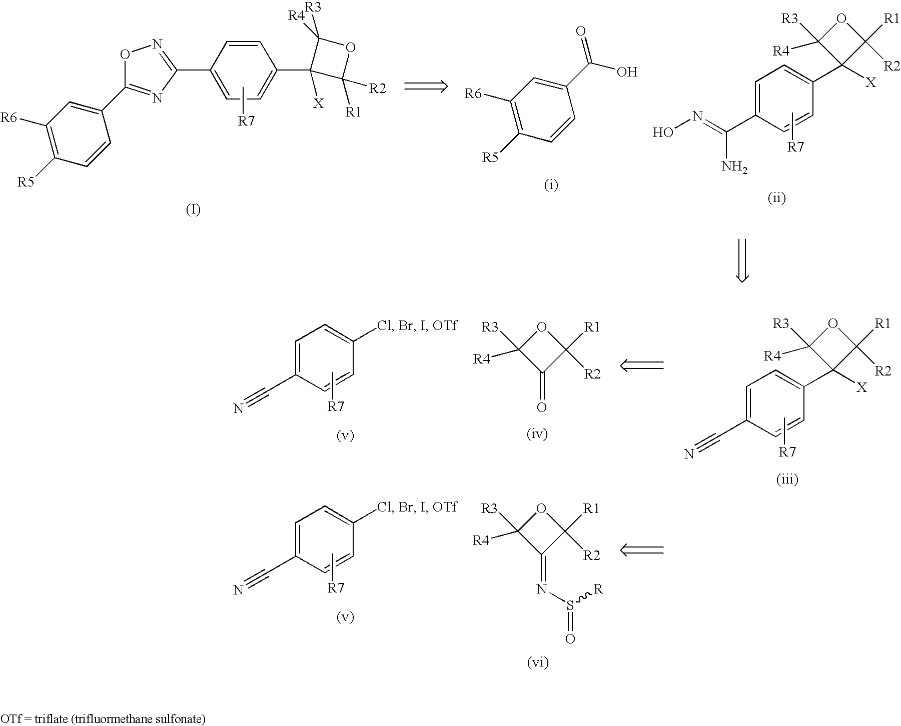

Reactions are typically carried out in a solvent such as methanol, ethanol, tetrahydrofuran, toluene, dichloromethane, 1,2-dichloroethane, N-methylpyrolidone, xylenes, ethyl acetate, diethyl ether, hexanes, cyclohexanes, dimethylformamide, acetone, dimethylsulfoxide, tert-butylmethyl ether. The above compounds may be isolated by using methods known to those skilled in the art (e.g. crystallization, silica gel chromatography, HPLC).

A compound of general formula (I) may be typically prepared via a 1,2,4 oxadiazole synthesis, i.e. by reacting a carboxylic acid according to formula (I) and an amidoxime according to formula (II) typically in the presence of a base (for example, Et₃N) and eventually a coupling agent (for example EDC/HOBt) and typically under heat. Said carboxylic acid of formula (I) may be commercially available or its preparation may be carried out by a method as suggested and/or as described in the literature. The coupling partner, i.e. an amidoxime according to formula (II), may be obtained from a nitrile of formula (iii) for example by reacting it with hydroxylamine for example in a solvent such as water.

In a compound of formula (I), wherein X═OH or O-Alkyl, the nitrile reagent of formula (iii) may be obtained by reacting an appropriate aryl halide or aryl triflate according to formula (v) with for example n-BuLi or magnesium, thereby causing a halogen exchange e.g. by virtue of generating a lithium or magnesium intermediate in situ, which is reacted with the corresponding keto oxetan in accordance to formula (Iv).

In a compound of formula (I), wherein X═NH₂ or NH-alkyl, the nitrile reagent of formula (iii) may be obtained by reacting an appropriate aryl halide or aryl triflate of formula (v) with for example n-BuLi or magnesium, thereby causing a halogen exchange e.g. by virtue of generating a lithium or magnesium intermediate in situ, which is reacted with the corresponding sulfinic amide in accordance to formula (vi), wherein R denotes an alkyl group.

A key building block is represented by a compound in accordance to formula (vi), being preferably used in the synthesis of the compounds of the present inventions, i.e. compounds of formula (I);

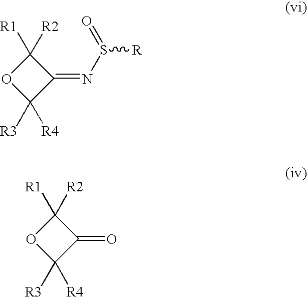

wherein the welded bond denotes the R-, or S-enantiomer or a racemic material, R1, R2, R3, R4 are as defined above and R represents an alkyl group.

A compound of formula (vi) may be obtainable e.g. by reacting a keto oxetan of general formula (Iv) and a corresponding sulfinamide (e.g an alkyl sulfinamide), wherein the R-, or S-enantiomer or the racemic material may be utilized, in presence of a dehydrating agent such as Ti(OEt)₄.

The above described intermediates of formulae (II), (iii), (iv) and (vi) may be chiral, e.g. S-enantiomer or R-enantiomer, or may be racemic, and may be in particular useful in the manufacturing of a compound of general formula (I). The chiral intermediates are typically suitable in controlling the chirality of the end-product, for example when the oxetan moiety in a compound of formula (I), (ii), (iii), (iv), and/or (vi) is asymmetric, for example when at least one but not all of R1, R2, R3 and/or R4 are different from hydrogen.

The following examples are illustrative of the invention, without any limitation. Concentration of solutions is typically carried out on a rotary evaporator under reduced pressure. Conventional flash chromatography is typically carried out on silica gel. Flash chromatography is also typically carried out using Biotage Flash Chromatography apparatus or Flashmaster instrument.

In a still further aspect the present invention relates to any aspect of the disclosed and described claims and/or examples individually, collectively or to any selections and/or any combinations thereof.

Abbreviations typically being used herein are:

TBME=tert.-butylmethyl ether

BOC=tert.-butyloxy carbonyl

BOC₂O=tert.-butyloxycarbonylanhydride

DMAP=N,N-dimethylaminopyridine

DMF=dimethylformamide

LiOH=lithium hydroxide

HCl=hydrochloric acid

THF=tetrahydrofuran

CH₂Cl₂=dichloromethane

RT=room temperature

NaOH=sodium hydroxide

HPLC=high pressure liquide chromatography

HOBt=hydroxybenzotriazole

EDC.HCl=1-ethyl-3-(3-dimethylaminopropyl)-carbodiimide hydrochloride

MS=mass spectroscopy

ES=electron spray

m/z=mass over charge number

### EXAMPLE 1

### 5-{3-[4-(3-Amino-oxetan-3-yl)-phenyl]-[1,2,4]oxadiazol-5-yl}-2-iso-propoxy-benzonitrile

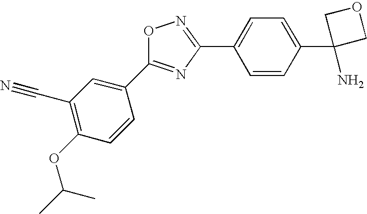

### a) 2-Methyl-propane-2-sulfinic acid oxetan-3-ylideneamide

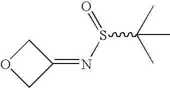

A 20% solution of oxetan-3-on (1 eq) in methylenechloride is diluted in THF. After that tert.butylsulfinamide (1.1 eq; racemate or optical pure (R)- or (S)-analog) and tetraethyl-orthotitanate (1.9 eq) is added with ice/water cooling under argon. After 16 hours at room temperature the reaction mixture is poured into a mixture of ethyl acetate and of brine. After filtering over Hyflo and extensive washing of the filter cake with ethyl acetate, the organic phase is dried over Na₂SO₄, filtered and concentrated. The resulting residue is purified on silica gel using methylenechloride/methanol 97.5/2.5 as eluent to give 2-methyl-propane-2-sulfinic acid oxetan-3-ylideneamide.

### b) 2-Methyl-propane-2-sulfinic acid [3-(4-cyano-phenyl)-oxetan-3-yl]-amide

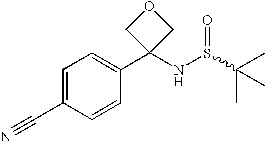

A solution of p-bromobenzonitrile (1 eq) in THF is cooled to −78° C. and subsequently n-butyl-lithium (1.6 M in hexane; 1.1 eq) is added under argon. To this reaction mixture a solution of 2-methyl-propane-2-sulfinic acid oxetan-3-ylideneamide (1.1 eq) in THF is added dropwise. After 1 hour at −78° C. the reaction mixture is allowed to warm up to room temperature and after 1 hour it is poured into saturated aqueous NaHCO₃ solution. The aqueous phase is 3 times extracted with ethyl acetate, the organic phase is dried over Na₂SO₄, filtered and concentrated. The resulting residue is purified on silica gel using methylenechloride/methanol 97.5/2.5 as eluent to give 2-methyl-propane-2-sulfinic acid [3-(4-cyano-phenyl)-oxetan-3-yl]-amide.

### c) [3-(4-Cyano-phenyl)-oxetan-3-yl]-carbamic acid tert-butyl ester

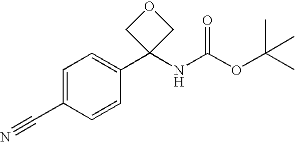

To a solution of 2-methyl-propane-2-sulfinic acid [3-(4-cyano-phenyl)-oxetan-3-yl]-amide in methylenechloride a solution of 2M HCl (in diethyl ether; 2 eq) is added. The resulting reaction mixture (precipitate formed) is diluted with cyclohexane and kept at room temperature for 40 minutes. Upon complete conversion the precipitate is filtered off, washed and dried.

The resulting filter cake is dissolved in dioxane followed by addition of 1M aqueous solution of NaOH (3 eq), BOC₂O (3.1 eq) and a catalytical amount of DMAP. The reaction mixture is stirred at room temperature for 16 hours, upon completion of the reaction, the mixture is diluted with water and extracted with ethyl acetate, the organic phase is dried over Na₂SO₄, filtered and concentrated. The resulting residue is purified on silica gel using methylenechloride/methanol 9/1 as eluent to give. [3-(4-cyano-phenyl)-oxetan-3-yl]-carbamic acid tert-butyl ester.

### d) {3-[4-(N-Hydroxycarbamimidoyl)-phenyl]-oxetan-3-yl}-carbamic acid tert-butyl ester

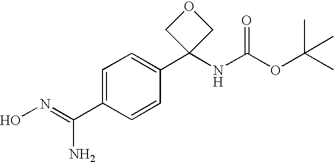

To a solution of [3-(4-cyano-phenyl)-oxetan-3-yl]-carbamic acid tert-butyl ester in THF is added at room temperature a 50% aqueous hydroxylamine solution in water (20 eq). The reaction mixture is stirred at room temperature for 48 hours. After removal of THF the reaction mixture is diluted with brine and extracted with ethyl acetate. The organic phase is dried over Na₂SO₄, filtered and concentrated. The resulting residue is suspended in a small amount diethyl ether, the resulting precipitate is filtered off to furnish {3-[4-(N-hydroxycarbamimidoyl)-phenyl]-oxetan-3-yl}-carbamic acid tert-butyl ester.

### e) (3-{4-[5-(3-Cyano-4-isopropoxy-phenyl)-[1,2,4]oxadiazol-3-yl]-phenyl}-oxetan-3-yl)-carbamic acid tert-butyl ester

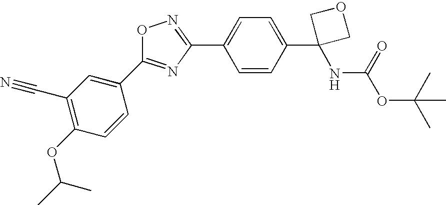

To a solution of 3-cyano-4-isopropoxy-benzoic acid (1 eq) in DMF is added under inert atmosphere EDC.HCl (1.1 eq) and HOBT (1.5 eq). The reaction mixture is then stirred at room temperature for 30 minutes. Then {3-[4-(N-hydroxycarbamimidoyl)-phenyl]-oxetan-3-yl}-carbamic acid tert-butyl ester (1.3 eq) is added to the reaction mixture, stirred at room temperature for 30 minutes, followed by heating at 95° C. for 6 hours. The reaction mixture is then concentrated to dryness, extracted with ethyl acetate/saturated aqueous NaHCO₃ solution. The organic phase is dried over Na₂SO₄, filtered, and concentrated all by routine procedural steps, and then purified using flash chromatography (methylenechloride/MeOH 9/1) to afford (3-{4-[5-(3-cyano-4-isopropoxy-phenyl)-[1,2,4]oxadiazol-3-yl]-phenyl}-oxetan-3-yl)-carbamic acid tert-butyl ester.

### f) 5-{3-[4-(3-Amino-oxetan-3-yl)-phenyl]-[1,2,4]oxadiazol-5-yl}-2-isopropoxy-benzonitrile (Example 1)

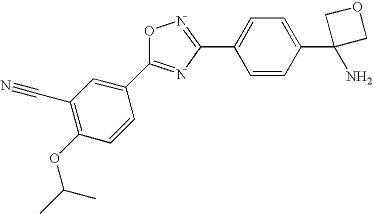

(3-{4-[5-(3-Cyano-4-isopropoxy-phenyl)-[1,2,4]oxadiazol-3-yl]-phenyl}-oxetan-3-yl)-carbamic acid tert-butyl ester is treated at 5° C. with neat TFA. After 10 minutes (clear solution) the reaction mixture is poured into a 0.02 M solution of HCl in diethyl ether. The resulting precipitate is filtered off, the resulting filter cake is washed with diethyl ether and suspended in ethyl acetate. The resulting suspension is refluxed under stirring and than cooled to 5° C. This procedure affords after filtering, washing with diethyl ether and drying 5-{3-[4-(3-amino-oxetan-3-yl)-phenyl]-[1,2,4]oxadiazol-5-yl}-2-isopropoxy-benzonitrile as hydrochloride salt.

### EXAMPLE 6

### 5-{3-[4-(3-Hydroxy-oxetan-3-yl)-phenyl]-[1,2,4]oxadiazol-5-yl}-2-ethoxy-benzonitrile

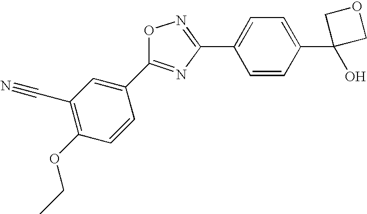

Example 6 is obtainable similar to the procedure recited in Example 1. However, only the following reaction steps and compounds are required:

Reaction step b) is carried out thereby using p-bromobenzonitrile and oxetan-3-on instead of 2-methyl-propane-2-sulfinic acid oxetan-3-ylideneamide followed by steps d) and e) thereby using 3-cyano-4-ethoxy-benzoic acid instead of 3-cyano-4-isopropoxy-benzoic acid.

### EXAMPLE 7

### 5-{3-[4-(3-Fluoro-oxetan-3-yl)-phenyl]-[1,2,4]oxadiazol-5-yl}-2-ethoxy-benzonitrile

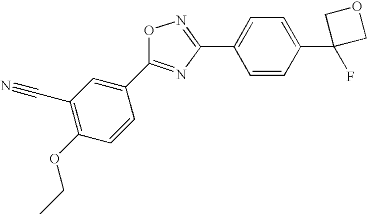

5-{3-[4-(3-Hydroxy-oxetan-3-yl)-phenyl]-[1,2,4]oxadiazol-5-yl}-2-ethoxy-benzonitrile (1 eq) is dissolved in methylenechloride and cooled down to −78° C. (using dry ice acetone mixture). A solution DAST (1.5 eq) is then added and the reaction mixture stirred at −78° C. for 10 minutes hours, then allowed to warm up to room temperature. The reaction is quenched with saturated aqueous NaHCO₃ solution. The organic phase is dried over Na₂SO₄, filtered, concentrated and purified on silica gel (ethyl acetate/cyclohexane ½ as mobile phase) to provide 5-{3-[4-(3-fluoro-oxetan-3-yl)-phenyl]-[1,2,4]oxadiazol-5-yl}-2-ethoxy-benzonitrile.

Table 1: Contains a number of examples being obtainable by the methods described above. The table further contains the corresponding molecular weights (MS ES⁺).

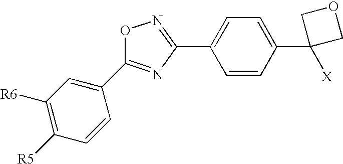

The variables R5, R6 and X are as defined below.

TABLE 1

| Ex | R5 | R6 | X | MS ES⁺ |
|---|---|---|---|---|
| 1 | i-propoxy | nitrile | amino | 377.0 |
| 2 | i-propoxy | nitrile | hydroxy | 378.2 |
| 3 | i-propoxy | acetyl | amino | 394.2 |
| 4 | ethoxy | nitrile | amino | 363.1 |
| 5 | i-propoxy | acetyl | hydroxy | 395.2 |
| 6 | ethoxy | nitrile | hydroxy | 364.1 |
| 7 | ethoxy | nitrile | fluoro | 366.1 |
| 8 | ethoxy | acetyl | amino | 380.2 |
| 9 | ethoxy | acetyl | hydroxy | 381.1 |

### Biology/Pharmacology

The compounds of formula I in free form or in pharmaceutically acceptable salt form, exhibit valuable pharmacological properties, e.g. as S1P1 receptor agonists, e.g. as indicated in vitro and in vivo tests and are therefore indicated for therapy.

### A. In Vitro

The compounds of formula I have agonistic activity to individual human S1P receptors and may be determined in the following assays:

A. In Vitro: GPCR Activation Assay Measuring GTP [γ-³⁵S] Binding to Membranes Prepared from CHO Cells Expressing Human EDG Receptors

S1P₁ (EDG-1) GTP [γ-³⁵S] assay: Homogenized membranes are prepared from CHO cell clones stably expressing a human EDG-1 N-terminal c-myc tag. Cells are grown in large culture dishes (500 cm²) to a confluence between 80 and 90%. The culture medium is removed and 20 mL ice-cold buffer A (10 mM HEPES, pH 7.4, 10 mM EDTA, complete protease inhibitor cocktail [1 tablet/50 mL]) added. The cells are harvested by scraping and the cell suspension centrifuged at 750×g for 10 min at 4° C. The pellet is resuspended in 10 mL ice-cold buffer B (20 mM HEPES, pH 7.4, 100 mM NaCl, 10 mM MgCl₂, 1 mM EDTA). The cell suspension is homogenized on ice, using a Polytron homogenizer at 25000 rpm at three intervals of 20 seconds each. Then the homogenate is centrifuged at 26900×g for 30 min at 4° C. and the membrane protein pellet resuspended by vortexing in cold buffer B. The protein concentration is determined using the Bio Rad Protein Assay and human IgG as standard. The volume of the membrane protein suspension is adjusted to a final concentration of about 2 mg protein/mL. The solution is then once again homogenized (Polytron) on ice at 25000 rpm for 20 seconds before being aliquoted and stored at −80° C.

Serial dilutions of compounds (stock in 10 mM DMSO) are prepared by first diluting the compounds in DMSO (1:10) followed by a 1:20 dilution into assay buffer (50 mM HEPES, pH 7.4, 5 mM MgCl₂, 1 mM CaCl₂, 1% fatty acid-free BSA). S1P (1 mM in DMSO/HCl) is diluted directly into assay buffer. The desired amount of membranes (1-5 μg/well) is diluted with assay buffer containing 10 μM GDP, 25 μg/mL Saponin and 5 mg/mL WGA-SPA beads. 9 μL of pre-diluted compound is placed into the bottom of a 96-well deep-well plate. 440 μL of the membrane-WGA-SPA bead slurry is added and the plate stirred for 15 minutes. Then twice 210 μL are transferred each into a 96-well Optiplate containing 15 μL GTP [γ-³⁵S] (4 nM in assay buffer). After incubation at room temperature for 120 minutes under constant shaking the plates are centrifuged for 10 minutes at 1000 g to pellet the membrane-SPA beads slurry. Then the plates are measured in a TOPcount NXT. Eight different concentrations of compound are used to generate a concentration response curve (using two data points per concentration) and the corresponding EC₅₀ value using the curve-fitting tool of GraphPad Prism.

S1P2, -3, -4, and -5 GTP [γ-³⁵S] binding assays are carried out in a comparable manner to the S1P1 GTP [γ-³⁵S] binding assay using membranes from CHO cells stably expressing c-terminal c-myc tagged or untagged receptors. For each membrane preparation, titration experiments are first run with S1P control to determine the optimal amount of membranes to be added per assay well.

Compounds of formula I are tested according to the above assay and show agonistic activity on S1P receptors, e.g. S1P1 receptors with an EC₅₀<1 μM. More particularly, compounds of formula I exhibit selectivity for the S1P1 receptor.

Biological agonistic activity in vitro (EC₅₀ in μmol/l) is shown in Table 2.

TABLE 2

| | S1P-1 GTPyS/EC₅₀ | S1P-5 GTPyS/EC₅₀ |
|---|---|---|
| Ex | [μmol I-1] | [μmol I-1] |
| 1 | 0.010 | 0.09 |
| 2 | 0.006 | 0.03 |
| 3 | 0.013 | 0.18 |
| 4 | 0.017 | 0.3 |
| 5 | 0.025 | 0.31 |
| 6 | 0.029 | 0.28 |

Moreover, compounds of formula I may exhibit selectivity for the S1P1 receptor compared to S1P2, S1P3 and S1P4, e.g. may at least be 20 times more selective for S1P1 compared to S1P2, S1P3 and S1P4.

Also typically, compounds of formula I may have a so-called dual selectivity for the S1P1 and S1P5 receptor over the other subtypes, namely S1P3 and S1P4. Said selectivity is typically around 5-20 (in terms of receptor selectivity). Such dual S1P1/S1P5 receptor agonists have valuable pharmacological efficacies.

The compounds of formula I in free form or in pharmaceutically acceptable salt form, exhibit still further valuable pharmacological properties such as for example an improved pharmacokinetic profile as being typically assessable by an ADME-study (ADME=absorption, distribution, metabolism and elimination). In particular, a compound in accordance to formula I, may typically exhibit a relatively fast elimination and hence may typically have an improved tolerability or less side effects.

### B. In Vivo: Screening Assays for Measurement of Blood Lymphocyte Depletion

Measurement of circulating lymphocytes: Lewis rats (male, 6-12 weeks old) are orally administered with 0.1 to 5 mg/kg of compound (4 ml/kg vehicle, e.g. in max. 2% DMSO/max. 2% PEG200/water or 0.5% methyl cellulose). A vehicle group is included as negative control.

Blood is collected from the sublingual vein at baseline (0 h) and 2, 6, 24 and 48 hours after administration under short isoflurane anesthesia. Whole blood samples are subjected to hematology analysis. Peripheral lymphocyte counts are determined using an automated analyzer. Two to four rats are used to assess the lymphocyte counts of each dose.

Administration of Example 1 at 0.1 to 5 mg/kg p.o leads to a dose-dependent reduction of peripheral lymphocyte counts. Maximal reduction is achieved at 6 h post-administration with 1 or 5 mg/kg. Lymphocyte counts fully (1 mg/kg) or partially recover back to baseline (5 mg/kg) within 48 hours.

### C. In Vivo: Animal Models of Experimental Autoimmune Encephalomyelitis (EAE)

The most widely used animal model for multiple sclerosis is experimental autoimmune encephalomyelitis (EAE), based on shared histopathological features with the human disease. EAE can be induced in susceptible animals by a single injection of antigen emulsified in complete Freund's adjuvant. A monophasic acute paralytic disease appears in susceptible rat strains, e.g., Lewis, Wistar rat, about 8-11 days post-sensitization. The symptomatic rats recover within the following 7 days, but in other species the attack is usually lethal. In the chronic progressive disease models rats undergo a chronic disease following the acute disease bout.

### Acute EAE Model

Female Lewis rats are injected intracutaneously in the hind-paws with 0.1 ml of a mixture of guinea pig spinal cord and complete Freund's adjuvant [3.5 g guinea pig spinal cord+3.5 ml 0.9% NaCl+105 mg M. tuberculosis H37Ra+7 ml CFA]. Symptoms of the disease (paralysis of the tail and both hind legs) develop within 9-10 days. The number of diseased animals as well as the time of onset of the disease is recorded. A minimum of five rats per group are used. Test compounds, e.g. Example 1 is administered daily, i.e. from days 0-13 days by oral gavage once or twice daily. In the absence of drug treatment symptoms of the disease (paralysis of the tail and both hind legs) usually develop within 8-11 days. Observable clinical symptom grades are typically:

1=loss of tail tonicity
2=weakness of one or both hind legs, or mild ataxia
3=severe ataxia or paralysis accompanied by urinary incontinence sometimes leading to death

In the above described test model compounds of the present invention, such as the compound of example 1, are typically active at a dose of 10 mg/kg b.i.d. and lead typically to the prevention of disease symptoms.

### Chronic-Progressive EAE Model (Therapeutic Treatment)

Induction of EAE in the DA rat is induced as previously described (Lorentzen et al, 1995, J. Neuroimmunol.; 63(2):193-205 and Adelmann et al, 1995, J. Neuroimmunol.; 63(1):17-27.). Briefly, antigen is prepared by homogenization of lyophilized bovine spinal cord in Arlacel A and DA rat brain and spinal cord homogenized in saline. These two mixtures (1:1) are then added to an equal volume of Complete Freund's Adjuvant (CFA) containing 16.6 mg/mL Mycobacterium tuberculosis H37Ra antigen. The total volume is homogenized to provide a consistent and well mixed adjuvant with antigen. All homogenization steps are carried out using a Polytron PT3100 homogenizer (Kinematica, Lucerne, Switzerland). Rats are immunized at 8-9 weeks of age, s.c. in the tail base with 200 μL of antigen/adjuvant mix (administering aprox. 19 mg bovine spinal cord, with 26 mg and 19 mg DA rat brain and spinal cord tissue, respectively), while anaesthetized by Isofluorane.

The resultant acute and subsequently chronic phase disease is typically evaluated using a numeric scale of progressive paralysis, such as:

0, no paralysis
1, loss of tail tonicity
2, hind limb weakening or ataxia
3, hind limb paralysis with or without urinary incontinence
4, hind limb and fore limb paralysis
5, moribund or death.

Clinical scores are usually evaluated on a daily basis. At the peak of clinical disease, and prior to initiation of the treatment regimens, animals are evenly distributed into the different groups based on onset and severity of clinical disease, to ensure comparability between each group in the initial acute disease leading to the chronic phase. Treatment of animals begins after the peak of clinical disease and continues daily until the end of the experiment. The test compound, i.e. a compound of the present invention, such as Example 1, is administered in e.g. 0.5% methyl cellulose (used also as the vehicle for the control group). Dosing volume is 5 mL/kg, adjusted to changes in body weight.

In the above described test model, usually all animals typically reach peak disease immediately before start of treatment, i.e. usually on day 12. The treatment of the said animal with a compound of the present invention, e.g. with Example 1, typically reduces the clinical scores to almost zero (<0.5), usually from day 15 post-immunization, i.e. usually after 4 days of treatment. Typically, said disease score(s) stay below 0.5 until treatment is discontinued (end of experiment).

### E. In Vivo: Animal Model of Experimental Autoimmune Uveitis (EAU)

S-antigen is produced as described elsewhere (e.g. Dorey C, et al. 1982, Ophthalm Res 14:249-255; or Wacker W B et al., 1977, J Immunol 119:1949-1958). The model of S-Antigen (S—Ag)-induced EAU is performed as previously described by Wacker (1977). Typically, female Lewis rats, 12 weeks of age are injected in the right footpad with 75 μg purified bovine retinal S—Ag. The antigen is dissolved in phosphate-buffered saline, and mixed 1+1 with Freund's Complete Adjuvant and Mycobacterium Tuberculosis H37Ra. The volume injected is 0.1 ml, containing 50 μl Freund Complete Adjuvant and 1.14 mg H37Ra. Starting at day 10 after injection, the eyes are inspected daily using an opthalmoscope (Heine, BETA 200).

The extent of ocular inflammation is scored in a semi-quantitative way by use of an opthalmoscope, following the scale:

0, no visible change
1, minimal change in the vasculature, some dilatation of iris and conjunctival blood vessels
2, moderate change, loss of vascular clearness, dilated iris and blood vessels, cloudy media
3, marked change, ocular protrusion, obscured pupil, pronounced loss of vascular architecture, some hemorrhage
4, severe change, marked ocular protrusion, complete loss of architecture, with diffuse hemorrhage.

In the above described model, untreated animals show an onset of the disease typically at day 9 and a maximum clinical score of 4 typically at day 13 post-immunization. Treatment with a compound of the present invention, such as Example 1, and typically dosed at 3 and 10 mg/kg bid p.o.; n=5, is usually initiated on day 7 post-immunization and is continued until day 18 (end of experiment). The treatment with a compound of the present invention, e.g. with Example 1, typically results in a dose-dependent protection from autoimmune uveitis.

The compounds of formula I are, therefore, useful in the treatment and/or prevention of diseases or disorders mediated by lymphocytes interactions, e.g. in transplantation, such as acute or chronic rejection of cell, tissue or organ allo- or xenografts or delayed graft function, graft versus host disease, autoimmune diseases, e.g. rheumatoid arthritis, systemic lupus erythematosus, hashimoto's thyroidis, multiple sclerosis, myasthenia gravis, diabetes type I or II and the disorders associated therewith, vasculitis, pernicious anemia, Sjoegren syndrome, uveitis, psoriasis, Graves opthalmopathy, alopecia greata and others, allergic diseases, e.g. allergic asthma, atopic dermatitis, allergic rhinitis/conjunctivitis, allergic contact dermatitis, inflammatory diseases optionally with underlying aberrant reactions, e.g. inflammatory bowel disease, Crohn's disease or ulcerative colitis, intrinsic asthma, inflammatory lung injury, inflammatory liver injury, inflammatory glomerular injury, atherosclerosis, osteoarthritis, irritant contact dermatitis and further eczematous dermatitises, seborrhoeic dermatitis, cutaneous manifestations of immunologically-mediated disorders, inflammatory eye disease, keratoconjunctivitis, myocarditis or hepatitis, e.g. acute or chronic hepatitis, ischemia/reperfusion injury, e.g. myocardial infarction, stroke, gut ischemia, renal failure or hemorrhage shock, traumatic shock, cancer, e.g. breast cancer, T cell lymphomas or T cell leukemias, nephrotic syndrome, infectious diseases, e.g. toxic shock (e.g. superantigen induced), septic shock, adult respiratory distress syndrome or viral infections, e.g. AIDS, viral hepatitis, e.g. hepatitis B or C, chronic bacterial infection, or neurodegenerative diseases, e.g. Alzheimer disease, amyotrophic lateral sclerosis or senile dementia. Examples of cell, tissue or solid organ transplants include e.g. pancreatic islets, stem cells, bone marrow, corneal tissue, neuronal tissue, heart, lung, combined heart-lung, kidney, liver, bowel, pancreas, trachea or oesophagus. For the above uses the required dosage will of course vary depending on the mode of administration, the particular condition to be treated and the effect desired.

In general, satisfactory results are indicated to be obtained systemically at daily dosages of about 1.0 to 20.0 mg/kg per body weight. An indicated daily dosage in the larger mammal, e.g. humans, is in the range from about 0.5 mg to about 500 mg, conveniently administered, for example, in divided doses up to four times a day or in retard form. Suitable unit dosage forms for oral administration comprise from ca. 0.1 to 50 mg active ingredient.

The compounds of formula I may be administered by any conventional route, in particular enterally, e.g. orally, e.g. in the form of tablets or capsules, or parenterally, e.g. in the form of injectable solutions or suspensions, topically, e.g. in the form of lotions, gels, ointments or creams, or in a nasal or a suppository form. Pharmaceutical compositions comprising a compound of formula I in free form or in pharmaceutically acceptable salt form in association with at least one pharmaceutical acceptable carrier or diluent may be manufactured in conventional manner by mixing with a pharmaceutically acceptable carrier or diluent.

The compounds of formula I may be administered in free form or in pharmaceutically acceptable salt form e.g. as indicated above. Such salts may be prepared in conventional manner and exhibit the same order of activity as the free compounds.

In accordance with the foregoing the present invention further provides:
1. A method for preventing or treating disorders or diseases mediated by lymphocytes, e.g. such as indicated above, in a subject in need of such treatment, which method comprises administering to said subject an effective amount of a compound of formula I or a pharmaceutically acceptable salt thereof;
2. A method for preventing or treating acute or chronic transplant rejection or T-cell mediated inflammatory or autoimmune diseases, e.g. as indicated above, in a subject in need of such treatment, which method comprises administering to said subject an effective amount of a compound of formula I or a pharmaceutically acceptable salt thereof;
3. A compound of formula I, in free form or in a pharmaceutically acceptable salt form for use as a pharmaceutical, e.g. in any of the methods as indicated under 1. or 2. above.
4. A pharmaceutical composition, e.g. for use in any of the methods as in 1. or 2. above, in particular comprising a compound of formula I in free form or pharmaceutically acceptable salt form, preferably in association with a pharmaceutically acceptable diluent or carrier therefore.
5. A compound of formula I or a pharmaceutically acceptable salt thereof for use in the preparation of a pharmaceutical composition for use in any of the method as in 1. or 2. above.

The compounds of formula I may be administered as the sole active ingredient or in conjunction with, e.g. as an adjuvant to, other drugs e.g. immunosuppressive or immunomodulating agents or other anti-inflammatory agents, e.g. for the treatment or prevention of allo- or xenograft acute or chronic rejection or inflammatory or autoimmune disorders, or a chemotherapeutic agent, e.g a malignant cell anti-proliferative agent. For example, the compounds of formula I may be used in combination with a calcineurin inhibitor, e.g. cyclosporin A or FK 506; a mTOR inhibitor, e.g. rapamycin, 40-O-(2-hydroxyethyl)-rapamycin, CC1779, ABT578, AP23573, biolimus-7 or biolimus-9; an ascomycin having immuno-suppressive properties, e.g. ABT-281, ASM981, etc.; corticosteroids; cyclophosphamide; azathioprene; methotrexate; leflunomide; mizoribine; mycophenolic acid or salt; mycophenolate mofetil; 15-deoxyspergualine or an immunosuppressive homologue, analogue or derivative thereof; a PKC inhibitor, e.g. as disclosed in WO 02/38561 or WO 03/82859, e.g. the compound of Example 56 or 70; a JAK3 kinase inhibitor, e.g. N-benzyl-3,4-dihydroxy-benzylidene-cyanoacetamide α-cyano-(3,4-dihydroxy)]N-benzylcinnamamide (Tyrphostin AG 490), prodigiosin 25-C (PNU156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate (also called CP-690,550), or a compound as disclosed in WO 04/052359 or WO 05/066156; immunosuppressive monoclonal antibodies, e.g., monoclonal antibodies to leukocyte receptors, e.g., MHC, CD2, CD3, CD4, CD7, CD8, CD25, CD28, CD40, CD45, CD52, CD58, CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists; or a chemotherapeutic agent, e.g. paclitaxel, gemcitabine, cisplatinum, doxorubicin or 5-fluorouracil; or an anti-infectious agent.

Where the compounds of formula I are administered in conjunction with other immunosuppressive/immunomodulatory, anti-inflammatory, chemotherapeutic or anti-infectious therapy, dosages of the co-administered immunosuppressant, immunomodulatory, anti-inflammatory, chemotherapeutic or anti-infectious compound will of course vary depending on the type of co-drug employed, e.g. whether it is a steroid or a calcineurin inhibitor, on the specific drug employed, on the condition being treated and so forth. In accordance with the foregoing the present invention provides in a yet further aspect:
6. A method as defined above comprising co-administration, e.g. concomitantly or in sequence, of a therapeutically effective non-toxic amount of a compound of formula I and at least a second drug substance, e.g. an immunosuppressant, immunomodulatory, anti-inflammatory or chemotherapeutic drug, e.g. as indicated above.
7. A pharmaceutical combination, e.g. a kit, comprising a) a first agent which is a compound of formula I as disclosed herein, in free form or in pharmaceutically acceptable salt form, and b) at least one co-agent, e.g. an immunosuppressant, immunomodulatory, anti-inflammatory, chemotherapeutic or anti-infectious agent. The kit may comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula I and a co-agent, are both administered to a patient simultaneously in the form of a single entity or dosage. The term “non-fixed combination” means that the active ingredients, e.g. a compound of formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

1. A compound formula I and/or a pharmaceutical acceptable salt thereof 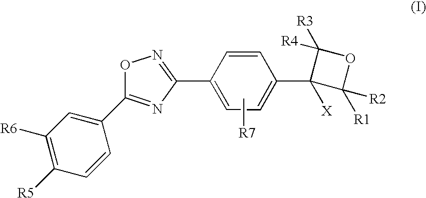
wherein
X is amino, alkylamino, hydroxyl, alkoxy or halo;
R1, R2, R3 and R4 are independently from each other H or lower alkyl;
R5 is alkoxy optionally substituted by halogen, C₃₋₆cycloalkoxy optionally substituted by halogen;
R6 is cyano, acyl, alkyl optionally substituted by halogen, or alkyl substituted by alkoxy; and
R7 is H, lower alkyl optionally substituted by halogen, lower alkoxy optionally substituted by halogen, or halogen.

2. A compound according to claim 1 wherein X is amino or hydroxyl.

3. A compound according to claim 1 wherein R1, R2, R3 and R4 are H.

4. A compound according to claim 1 wherein R5 is alkoxy optionally substituted by halogen, or C₃₋₆cycloalkoxy optionally substituted by halogen.

5. A compound according to claim 1 wherein R6 is cyano or acyl.

6. A compound of formula I according to claim 1 or a pharmaceutically acceptable salt thereof for use as a pharmaceutical.

7. A pharmaceutical composition comprising a compound of formula I according to claim 1 or a pharmaceutically acceptable salt thereof, in association with a pharmaceutically acceptable diluent or carrier therefore.

8. A compound of formula I according to claim 1 or a pharmaceutically acceptable salt thereof for use in preventing or treating disorders or diseases mediated by lymphocytes.

9. A pharmaceutical combination comprising a) a first agent which is a compound of formula I according to claim 1, in free form or in pharmaceutically acceptable salt form, and b) at least one co-agent which is an immunosuppressant, immunomodulatory, anti-inflammatory, chemotherapeutic or anti-infectious agent.

10. A method for preventing or treating disorders or diseases mediated by lymphocytes, in a subject in need of such treatment, which method comprises administering to said subject an effective amount of a compound of formula I according to claim 1 or a pharmaceutically acceptable salt thereof.

11. A process for the manufacture a compound in accordance to formula (I), wherein the variables are as defined in claim 1, 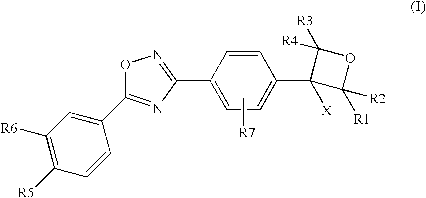
which process is characterized by reacting an oxetan-3-one of formula (Iv), wherein the variables are as defined in claim 1, 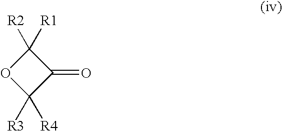
with alkyl sulfonamide (e.g. with tert.butylsulfinamide), wherein the R-, or S-enantiomer or the racemic material may be utilized, with a dehydrating agent, for example with tetraethyl-orthotitanate, into alkyl-2-sulfinic acid oxetan-3-ylideneamide in accordance to formula (vi), wherein the welded bond denotes the R-, or S-enantiomer or a racemic material, 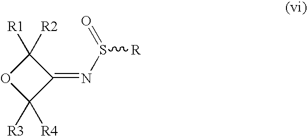
which may be reacted with e.g. 4-halo-benzonitrile and a base such as butyl lithium to provide the 4-cyano-compound of formula (iiia) 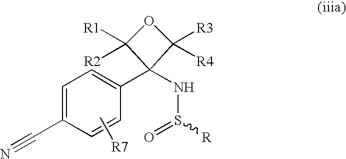
which may be converted, e.g. by consecutive reactions with an acid and then with BOC₂O, to the carbamic acid tert-butyl ester in accordance to formula (iiib), 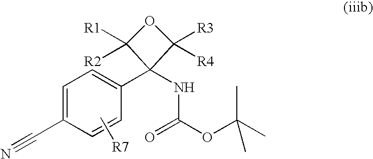
which may be converted for example with an excess of hydroxylamine, e.g. in aqueous solution, into the hydroxycarbamimidoyl-phenyl-oxetan-3-yl-carbamic acid tert-butyl ester of formula (iia), 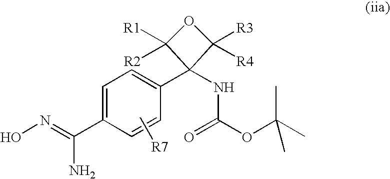
which may be cyclized typically under amino acid coupling conditions, such as EDC.HCl/HOBT, with a benzoic acid, such as for example R5- and R6-disubstituted benzoic acid, to the—[1,2,4]oxadiazol compound of formula (Ia), 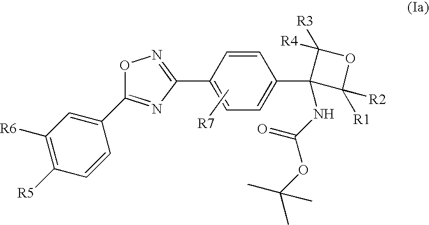
which may be then be converted with an acid such as neat TFA into the final product of formula (Ic).

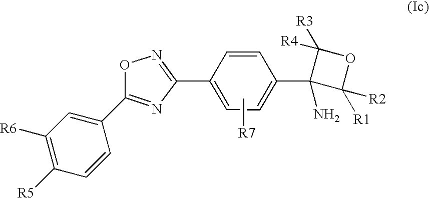

12. An Intermediate compound as disclosed in the previous claim and in accordance to the formulae (iia), (iiia), (iiib), and/or (vi), wherein the variables have the definitions as described hereinbefore, substantially as described in the working examples, claims and/or in the description, being in particular useful in the preparation of a compound of formula (I) in accordance to claim 1, wherein the variables are as provided in claim 1.

13. A compound of formula (vi), 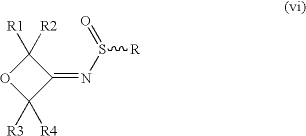
wherein R1, R2, R3 and R4 are independently from each other H or lower alkyl, R is alkyl, and wherein the welded bond denotes the R-, or S-enantiomer or the racemic material, said compound of formula (vi) being in particular useful as an intermediate in the preparation of a compound of general formula (I).

14. A compound of claim 13, wherein R1, R2, R3 and R4 are hydrogen, R is t-butyl, wherein the welded bond denotes the R-, or S-enantiomer or a racemic material, which is compound of formula (vi a). [chemical structure]
